(19) United States
(12) Patent Application Publication
(10) Pub. No.: US 2004/0241661 A1
(43) Pub. Date: Dec. 2, 2004

(54) Pseudo single color method for array assays

(76) Inventor: Arindam Bhattacharjee, Andover, MA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/449,136

(22) Filed: May 29, 2003

(51) Int. Cl. C12Q 1/68; G06F 19/00; G01N 33/48; G01N 33/50

(52) U.S. Cl. 435/6; 702/20

(57) ABSTRACT

Methods of determining the amount of an analyte in a mixture of analytes are provided. The methods involve contacting a sample of analytes that is labeled with two or more distinguishably detectable labels with a probe for the analyte, and determining the amounts of the two or more distinguishably detectable labels bound with the probe. In certain embodiments, the methods include averaging the amounts of the two or more labels in order to determine the amount of analyte in the sample. Kits are provided for performing the invention. The subject invention finds use in a variety of different applications, including gene expression analysis, DNA sequencing, mutation detection and other genomics and proteomics applications.

### FIELD OF THE INVENTION

The field of this invention is arrays, particularly nucleic acid microarrays.

### BACKGROUND OF THE INVENTION

In nucleic acid sequencing, mutation detection, proteomics, and gene expression analysis, there is a growing emphasis on the use of high density arrays of immobilized nucleic acid or polypeptide probes. Such arrays can be prepared by a variety of approaches, e.g., by depositing biopolymers, for example, cDNAs, oligonucleotides or polypeptides on a suitable surface, or by using photolithographic techniques to synthesize biopolymers directly on a suitable surface. Arrays constructed in this manner are typically formed in a planar area of between about 4-100 mm², and can have densities of up to several thousand or more distinct array members per cm².

In use, an array surface is contacted with a labeled sample containing target analytes (usually nucleic acids or proteins) under conditions that promote specific, high-affinity binding of the analytes in the sample to one or more of the probes present on the array. The goal of this procedure is to quantify the level of binding of one or more probes of the array to labeled analytes in the sample. Typically, the analytes in the sample are labeled with a detectable label such as a fluorescent tag, and quantification of the level of fluorescence associated with a bound probe represents a direct measurement of the level of binding. In turn, this measurement of binding represents an estimate of the abundance of a particular analyte in the sample. A variety of biological and/or chemical compounds may be used as detectable labels in the above-described arrays (See, e.g., Wetmur, J. Crit Rev Biochem and Mol Bio 26:227, 1991; Mansfield et al., Mol Cell Probes. 9:145-56, 1995; Kricka, Ann Clin Biochem. 39:114-29, 2002).

Such arrays are commonly used to perform nucleic acid hybridization assays. Generally, in such a hybridization assay, labeled single-stranded analyte nucleic acid (e.g. polynucleotide target) is hybridized to an immobilized complementary single-stranded nucleic acid probe. Complementary nucleic acid probe binds the labeled target polynucleotide, and the presence of the labeled target polynucleotide of interest is detected and quantified.

However, despite their popularity, array assays have several drawbacks that lead to unreliable results. One drawback is that stearic hindrance between bulky dye labels (e.g., cyanine-dye labeled nucleotides) results in preferential incorporation of labels into certain analytes during sample labeling. For example, if a certain polynucleotide in a sample has a position that contains a contiguous stretch of “T” nucleic acids, and the labeled nucleotide is a modified “T” nucleotide bonded to a bulky label, a polymerase may not be able to incorporate a contiguous stretch of labeled T's at that position because the bulky labels stearically interfere with the activity of the polymerase. This frequently leads to stalling of the polymerase during labeling of a particular nucleic acid species and leads to a disproportionately low level of a labels associated with that species. Another drawback for nucleic acid array assays is that some nucleic acid species in a nucleic acid sample may consist of significantly more of a certain type of nucleotide than other nucleic acid species in the sample. Such nucleic acid species may be disproportionately heavily labeled if it is labeled using a label that is conjugated to that nucleotide. Furthermore, certain labels are difficult to detect over background signals caused by non-specific binding or binding of unincorporated labels to a probe or to the surface of an array substrate. These problems may be complicated by the fact that certain labels cause more background signal than other labels, and binding may not proceed at the same rate across an array of probes. Furthermore, some dyes are more labile than others, for example, to ozone. Signal bias leads to an under- or over-estimation of a certain analytes in a sample of analytes, and, as such, if the bias is uncorrected, the bias may lead to erroneous results.

Accurate estimation of the level of a particular analyte in a mixture of analytes is therefore complicated, and typically involves algorithms for background signal calculation, and statistical analysis of several identical array assays.

A common approach to correct signal bias is to provide a global reference, such as, in the case of nucleic acid array assays, by measuring the amounts of probe present in each element on an array, or by measuring the levels of binding of the probes on the array to a control sample. The latter of these approaches is currently the approach of choice, and, in the case of nucleic acid array assays, usually involves methods of hybridizing two different distinguishably labeled nucleic acid samples with an array of probes. These methods usually result in two sets of data: an experimental set of data, and a reference set of data to which the experimental data is compared. In these methods, the levels of an analyte within a sample is usually expressed as a ratio that describes the relative levels of a analyte in two samples. While these approaches have been quite successful in reducing signal bias caused by variable amounts of probes immobilized on the arrays, they have failed to correct the other signal biases described above.

As such, there is a continued need for reliable methods for estimating the levels of analyte in a sample, such as in methods of determining the amounts of nucleic acid in a nucleic acid sample. This invention meets this, and other, needs.

Relevant Literature

Publications of interest include: U.S. Pat. Nos. 4,868,105; 5,124,246; 5,563,034; and 5,681,702; PCT publication WO 98/24933; Bilban et al, Curr Issues Mol Biol. 4:57-64, 2002, Freeman et al, Biotechniques 29:1042-6 and 1048-55, 2000, DeRisi et al. Science 278:680-686, 1997, Quackenbush et al, Nat Genet. 32 Suppl:496-501, 2002, Bilban et al Curr Issues Mol Biol. 4:57-64, 2002, Finkelstein et al, Plant Mol Biol. 48(1-2):119-31, 2002, and Hegde et al, Biotechniques. 29:548-554, 2000.

### SUMMARY OF THE INVENTION

Methods of determining the amount of an analyte in a mixture of analytes are provided. The methods involve contacting a sample of analytes that is labeled with two or more distinguishably detectable labels with a probe for the analyte, and determining the amounts of the two or more distinguishably detectable labels bound with the probe. In certain embodiments, the methods include averaging the amounts of the two or more labels in order to determine the amount of analyte in the sample. Kits are provided for performing the invention. The subject invention finds use in a variety of different applications, including gene expression analysis, DNA sequencing, mutation detection and other genomics and proteomics applications.

### BRIEF DESCRIPTION OF THE DRAWINGS

FIG. 1 is a compilation of two bar graphs showing the mean of either unnormalized or normalized data. Normalization refers to a adjustment to the distribution of the original signal intensities (un-normalized) of datapoints in two color channels such that the range and the spread of the data is similar in both.

FIG. 2 is a scatter plot showing coefficient of variation for several data points obtained using the subject methods.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

A “biopolymer” is a polymer of one or more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups. Biopolymers include polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Biopolymers include DNA (including cDNA), RNA, oligonucleotides, and PNA and other polynucleotides as described in U.S. Pat. No. 5,948,902 and references cited therein (all of which are also incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (e.g., a single amino acid or nucleotide with two linking groups one or both of which may have removable protecting groups).

An “array,” includes any one, two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination which will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

A “scanner” is device for evaluating arrays. In scanners, an optical light source, particularly a laser light source, generates a light which is focused on the array and sequentially illuminates surface regions of known location (for example, a point or line) on an array substrate. The resulting signals from the surface regions are collected either employing the same lens used to focus the light onto the array or using a separate lens positioned to one side of the lens used to focus the onto the array. The collected signals may be then transmitted through appropriate spectral filters, to an optical detector. A recording device, such as a computer memory, records the detected signals and builds up a scan file of intensities as a function of position, or time as it relates to the position. In the case of spot illumination, such intensities, as a function of position, are typically referred to in the art as “pixels”. Biopolymer arrays are often scanned and/or scan results are often represented at 5 or 10 micron pixel resolution. To achieve the precision required for such activity, components such as the lasers must be set and maintained with particular alignment. Scanners may be bi-directional, or unidirectional, as is known in the art.

The scanner typically used for the evaluation of arrays includes a scanning fluorometer. A number of different types of such devices are commercially available from different sources, such as such as Perkin-Elmer, Agilent, or Axon Instruments, etc., and examples of typical scanners are described in U.S. Pat. Nos: 5,091,652; 5,760,951, 6,320,196 and 6,355,934.

As used herein, the terms “hybridization,” “hybridizing” and “binding” may be used interchangeably. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to the nucleotides in another sequence, the more stringent the conditions can be for hybridization and the more specific will be the binding of the two sequences. Increased stringency is achieved by elevating the temperature, increasing the ratio of co-solvents, lowering the salt concentration, and the like. Hybridization processes and conditions are described by Sambrook, J. et al., (Molecular Cloning: A Laboratory Manual, Cold Spring Harbor Laboratory Press, Cold Spring Harbor, 2^{nd} Ed., 1989, vol. 1-3). Conditions for hybridization typically include high ionic strength solution, controlled temperature, and the presence of carrier DNA and detergents and divalent cation chelators, all of which are well known in the art.

As used herein, the term “specific hybridization” refers to those occurrences in which a segment of an oligonucleotide probe preferentially hybridizes with a segment of a selected polynucleotide, as intended. The use of the term “hybridizes” is not meant to exclude non Watson-Crick base pairing.

Specific hybridization usually occurs under conditions of stringent hybridization. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 533 SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art and may also be employed to identify nucleic acids of this particular embodiment of the invention.

The term “sample” refers to a sample derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). The sample may contain a single- or double-stranded nucleic acid molecule which includes a target nucleotide sequence and may be prepared for hybridization analysis by a variety of means, e.g., using proteinase K/SDS, chaotropic salts, or the like. In many embodiments, the sample is a complex sample containing at least about 10², 5×10², 10³, 5×10³, 10⁴, 5×10⁴, 10⁵, 5×10⁶, 10⁶, 5×10⁶, 10⁷, 5×10⁷, 10⁸, 10⁹, ¹⁰, 10¹¹, 10¹² or more “target” species.

The terms “target” “target molecule” and “analyte” are used herein interchangeably and refer to a known or unknown molecule in a sample, which will specifically bind, e.g., hybridize, to a molecular probe on a substrate surface if the target molecule and the molecular probe contain complementary regions, i.e., if they are members of a specific binding pair. In general, the target molecule is a biopolymer, i.e., an oligomer or polymer such as an oligonucleotide, a peptide, a polypeptide, a protein, and antibody, or the like. In this case, a “target” is referenced as a moiety in a mobile phase (typically fluid), to be detected by a “probe” which is bound to a substrate. However, either of the “target” or “probe” may be the one which is to be evaluated by the other (thus, either one could be an unknown mixture of analytes, e.g., polynucleotides, to be evaluated by binding with the other).

As used herein, the terms “target region” or “target nucleotide sequence” may be used interchangeably, and refers to a sequence of nucleotides to be identified, e.g., an analyte target nucleic acid, usually existing within a portion or all of a polynucleotide, usually a polynucleotide analyte. Target nucleic acids are found in a sample. The identity of the target nucleotide sequence generally is known to an extent sufficient to allow preparation of various probe sequences hybridizable with the target nucleotide sequence. The term “target sequence” refers to a sequence with which a probe will form a stable hybrid under desired conditions. The target sequence generally contains from about 30 to 5,000 or more nucleotides, preferably about 50 to 1,000 nucleotides. The target nucleotide sequence is generally a fraction of a larger molecule or it may be substantially the entire molecule such as a polynucleotide as described above. The minimum number of nucleotides in the target nucleotide sequence is selected to assure that the presence of a target polynucleotide in a sample is a specific indicator of the presence of polynucleotide in a sample. The maximum number of nucleotides in the target nucleotide sequence is normally governed by several factors: the length of the polynucleotide from which it is derived, the tendency of such polynucleotide to be broken by shearing or other processes during isolation, the efficiency of any procedures required to prepare the sample for analysis (e.g. transcription of a DNA template into RNA) and the efficiency of detection and/or amplification of the target nucleotide sequence, where appropriate.

Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G=U or U=G base pairs. Thus, two sequences need not have perfect homology to be “complementary” under the invention, and in most situations two sequences are sufficiently complementary when at least about 85% (preferably at least about 90%, and most preferably at least about 95%) of the nucleotides share base pair organization over a defined length of the molecule.

The term “substrate” is used interchangeably herein with the terms “support” and “solid substrate,” and denotes any solid support suitable for immobilizing one or more probes.

A “probe” is a biopolymer that is usually immobilized on a substrate, and forms a feature, or element, on an array. Probes, like targets, may be nucleic acids, antibodies, polypetides, and the like. Nucleic acid probes are hybridizable in that they have a nucleotide sequence that can hybridize to a target nucleic acid, if present, under suitable hybridization conditions. In most embodiments, a probe is a single stranded nucleic acid of at least about 15 bp, at least about 20 bp, at least about 30 bp, at least about 50 bp, at least about 100 bp, at least about 200 bp, at least about 500 bp, at least about 800 bp, at least about 1 kb, at least about 1.6 kb, at least about 2 kb, at least about 3 kb or at least about 5 kb or more in length.

### DETAILED DESCRIPTION OF THE INVENTION

Methods of determining the amount of an analyte in a mixture of analytes are provided. The methods involve contacting a sample of analytes that is labeled with two or more distinguishably detectable labels with a probe for the analyte, and determining the amounts of the two or more distinguishably detectable labels bound with the probe. In certain embodiments, the methods include averaging the amounts of the two or more labels in order to determine the amount of analyte in the sample. Kits are provided for performing the invention. The subject invention finds use in a variety of different applications, including gene expression analysis, DNA sequencing, mutation detection and other genomics and proteomics applications.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In further describing the subject invention, the subject methods of determining the amount of an analyte in a sample are described first, followed by a review of applications that utilize the subject methods. Finally, kits and systems for use in practicing the subject methods are described.

Methods of Determining the Amount of an Analyte in a Sample

The subject invention provides a method of determining the amount of an analyte, e.g. a polypeptide or a nucleic acid, in a sample of analytes that is labeled with at least a first and a second distinguishable detectable label. In general, the method includes the following steps a) contacting a probe for an analyte to the sample under conditions sufficient for specific binding to occur between the probe and the analyte; and b) identifying the amount of the first and second labels in the resultant analyte/probe complex, thereby determining the amount of the analyte in the sample.

In many embodiments, the analyte is a nucleic acid, and the method includes the steps of: a) contacting a probe for a nucleic acid to a sample of nucleic acids under conditions sufficient for duplex nucleic acids to be produced between the probe and the nucleic acid, and b) identifying the amount of the first and second labels in the resultant duplex nucleic acid.

In certain embodiments nucleic acid sample may be a sample of RNA or DNA, either single stranded or double stranded. In many embodiments, the nucleic acid is extracted from a source (e.g., a cell, group of cells, tissue, culture, etc.) of interest, and includes RNA (e.g., unspliced RNA or mRNA, etc.), or DNA (e.g., genomic DNA of a nucleus or organelle, etc.). In certain embodiments, the sample is a genetic copy of the nucleic acid extracted from a source, such as cDNA, amplified DNA or RNA, or a nucleic acid that contains modified nucleotide residues (e.g., amino-allyl nucleotides). Nucleic acid compositions suitable for labeling in the subject methods are well known in the art, and their further description may be found in several publications, including Brumbaugh et al (Proc Natl Acad Sci U S A 85, 5610-4, 1988), Hughes et al. (Nat Biotechnol 19, 342-7, 2001), Eberwine et al (Biotechniques. 20:584-91, 1996), Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.).

In many embodiments, the sample contains labeled analytes, e.g. nucleic acids, where individual analyte molecules within the sample are labeled with at least two, (e.g., two, three, four, five, six, seven or eight or more) detectably distinguishable labels. At least 2, at least about 4, at least about 6, at least about 8, at least about 10, at least about 15, at least about 20, at least about 25, at least about 30, or at least about 40 or at least about 50 or more of each distinguishable detectable label may associated with a single analyte molecule. In certain embodiments, however, particularly those that involve separately labeling two portions of the same sample and mixing the labeled portions together to make a labeled sample, individual analyte molecules within the sample may be labeled with only one type of label.

Labels of interest include directly detectable and indirectly detectable non-radioactive labels such as fluroscent dyes. Directly detectable labels are those labels that provide a directly detectable signal without interaction with one or more additional chemical agents. Examples of directly detectable labels include fluorescent labels. Indirectly detectable labels are those labels which interact with one or more additional members to provide a detectable signal. In this latter embodiment, the label is a member of a signal producing system that includes two or more chemical agents that work together to provide the detectable signal. Examples of indirectly detectable labels include biotin or digoxigenin, which can be detected by a suitable antibody coupled to a fluorochrome or enzyme, such as alkaline phosphatase. In many preferred embodiments, the label is a directly detectable label. Directly detectable labels of particular interest include fluorescent labels.

Fluorescent labels that find use in the subject invention include a fluorophore moiety. Specific fluorescent dyes of interest include: xanthene dyes, e.g. fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R⁶G⁶ or G⁶), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc.

As mentioned above, the labels used in the subject methods are distinguishable, meaning that the labels can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), and POPRO3 TOPRO3 (Molecular Probes, Eugene, Oreg.). Further suitable distinguishable detectable labels may be found in Kricka et al. (Ann Clin Biochem. 39:114-29, 2002).

In general, at least two distinguishable labels are covalently attached to analytes in a sample. Means for labeling proteins and nucleic acids are generally well known in the art (e.g. Brumbaugh et al Proc Natl Acad Sci U S A 85, 5610-4, 1988; Hughes et al. Nat Biotechnol 19, 342-7, 2001, Eberwine et al Biotechniques. 20:584-91, 1996, Ausubel, et al, Short Protocols in Molecular Biology, 3rd ed., Wiley & Sons, 1995 Sambrook, et al, Molecular Cloning: A Laboratory Manual, Third Edition, 2001 Cold Spring Harbor, N.Y. and DeRisi et al. Science 278:680-686, 1997; Patton W F. Electrophoresis. 2000 21:1123-44; MacBeath G. Nat Genet. 2002 32 Suppl:526-32; and Biotechnol Prog. 1997 13:649-58). These means usually involve either direct chemical modification of the analyte, or a labeled nucleotide that is incorporated into a nucleic acid by nucleic acid replication, e.g., using a polymerase.

Chemical modification methods for labeling a nucleic acid sample usually include incorporation of a reactive nucleotide into a nucleic acid, e.g., an amine-allyl nucleotide derivative such as 5-(3-aminoallyl)-2′-deoxyuridine 5′-triphosphate, using an RNA-dependent or DNA-dependent DNA or RNA polymerase, e.g., reverse transcriptase or T7 RNA polymerase, followed by chemical conjugation of the reactive nucleotide to a label, e.g. a N-hydroxysuccinimdyl of a label such as Cy-3 or Cy5 to make a labeled nucleic acids (Brumbaugh et al Proc Natl Acad Sci U S A 85, 5610-4, 1988 and Hughes et al. Nat Biotechnol 19, 342-7, 2001). Such chemical conjugation methods may be combined with RNA amplification methods (e.g. those of Eberwine et al Biotechniques. 20:584-91, 1996), to produce labeled DNA or RNA.

Suitable labels may also be incorporated into a sample by means of nucleic acid replication, where modified nucleotides such as modified deoxynucleotides, ribonucleotides, dideoxynucleotides, etc., or closely related analogues thereof, e.g. a deaza analogue thereof, in which a moiety of the nucleotide, typically the base, has been modified to be bonded to the label. Modified nucleotides are incorporated into a nucleic acid by the actions of a nucleic acid-dependent DNA or RNA polymerases, and a copy of the nucleic acid in the sample is produced that contains the label. Methods of labeling nucleic acids by a variety of methods, e.g., random priming, nick translation, RNA polymerase transcription, etc., are well generally known in the art (see, e.g., Ausubel, et al, Short Protocols in Molecular Biology, 3rd ed., Wiley & Sons, 1995 Sambrook, et al, Molecular Cloning: A Laboratory Manual, Third Edition, 2001 Cold Spring Harbor, N.Y. and DeRisi et al. Science 278:680-686, 1997).

In most embodiments of the invention, an analyte sample is labeled using a mixture of labels. In other words, two or more distinguishably detectable labels are mixed together, usually in a single vessel or tube, sometimes in equal proportions, in a single labeling reaction for a sample. The two or more labels may be for the same nucleotide e.g. “T” or “U”, or a mixture of two, three or four nucleotides. In certain embodiments, however, if the samples are identical (e.g. they are two portions of a sample, or two nucleic acids samples made from the same source), the samples may be labeled separately and combined to make a labeled sample. As such, the subject methods do not involve labeling two different samples (e.g. samples from two different tissues, times, or conditions), each with distinguishable label, and mixing the samples together.

Once labeled, the sample is usually applied to a substrate that includes at least one probe, and incubated under conditions suitable for an analyte/probe complex, e.g. a nucleic acid duplex (i.e. a RNA/RNA, DNA/RNA, or DNA/DNA duplex) to be formed between a probe and a labeled analyte in the sample, if such a labeled analyte is present. In other words, the labeled analyte sample is incubated with a substrate that contains at least one probe under conditions suitable for binding of the labeled analyte to the probe. In certain embodiments, the substrate that includes the probe is an array of probes, where each probe is contained in a feature of the array, and where an array includes at least about 20, at least about 50, at least about 100, at least about 200, at least about 500, at least about 1,000, at least about 2,000, at least about 5,000, at least about 10,000, at least about 20,000, at least about 50,000, usually up to about 100,000 or more features. Arrays used in the subject methods may have known amounts of probes present in a feature of the array. For example, if the concentration of a probe in a solution of probe to be deposited as known, and, the volume of the probe solution that is deposited in a feature is known, an amount of probe present in a feature of an array may be known.

After incubation, labeled sample that is not bound with a probe is typically washed away from the substrate, and the substrate, now including the labeled analyte/probe duplexes, is scanned. The amount of each label associated with features of the array (each feature containing, e.g., a target analyte/probe complex or a probe if no target analyte is present) is then determined. In most embodiments, the substrate is scanned in two channels corresponding to the distinguishing features of the probes, such that the amounts of each label associated with each feature is determined independently (i.e. without interference) from other labels. In certain embodiments, scanning results in two scans, one for each channel, and usually represents a pixilated image of the substrate that reflects the amount of label associated with the features of the substrate. For example, each pixel of the image is accorded a signal level that represents the level of brightness of the label signal. As mentioned above, scanning methods are well known in the art (e.g., DeRisi et al. Science 278:680-686, 1997), and several suitable scanners are commercially available from Perkin-Elmer, Agilent, or Axon Instruments, etc., and are described in U.S. Pat. Nos: 5,091,652; 5,760,951, 6,320,196 and 6,355,934), the disclosures of which are herein incorporated by reference.

Feature Extraction and Data Analysis

Feature extraction describes the method by which numerical data is obtained from an array. In general feature extraction methods involve identifying a feature (usually corresponding to a probe) on a scan of a hybridized array, and measuring the amount of label (e.g., fluorescence) that is associated with the feature. In most embodiments, feature extraction methods provide a numerical figure for chosen features in each of the two or more scans of an array. Several commercially available programs perform feature extraction on microarrays, such as IMAGINE® by BioDiscovery (Marina Del Rey, Calif.) Stanford University's “ScanAlyze” Software package, Microarray Suite of Scanalytics (Fairfax, Va.), “DeArray” (NIH); PATHWAYS® by Research Genetics (Huntsville, Ala.); GEM tools® by Incyte Pharmaceuticals, Inc., (Palo Alto, Calif.); Imaging Research (Amersham Pharmacia Biotech, Inc., Piscataway, N.J.); the RESOLVER® system of Rosetta (Kirkland, Wash.) and the Feature Extraction Software of Agilent Technologies (Palo Alto, Calif.). Such commercially available programs may be adapted or modified to perform the subject methods.

Numerical data corresponding to the amount of label associated with features of an array are produced using feature extraction software, as described above. In most embodiments, at least two groups of data are produced (i.e., two, three, four, five, or six or more groups of data are produced) each set corresponding to a distinguishable label. Each numerical value associated with an amount of signal for a feature may also be associated with other numerical values pertaining to the quality of the label signal from the feature (e.g. values associated with the variation in pixel intensity for a feature), as is commonly provided by the commercially available programs described above. Amounts of signal may be measured as an quantitative (i.e. absolute) value of signal, or a qualitative (e.g. relative) value of signal, as is known in the art.

In most embodiments of the invention, the data groups are globally normalized to each other, and/or normalized to data obtained from controls (e.g., internal controls produce data that are predicted to equal in value in all of the data groups). Normalization generally involves multiplying each numerical value for one data group by a value that allows the direct comparison of those amounts to amounts in a second data group. Several normalization strategies have been described (Quackenbush et al, Nat Genet. 32 Suppl:496-501, 2002, Bilban et al Curr Issues Mol Biol. 4:57-64, 2002, Finkelstein et al, Plant Mol Biol.48(1-2):119-31, 2002, and Hegde et al, Biotechniques. 29:548-554, 2000). Specific examples of normalization suitable for use in the subject methods include linear normalization methods, non-linear normalization methods, e.g., using lowess local regression to paired data as a function of signal intensity, signal-dependent non-linear normalization, qspline normalization and spatial normalization, as described in Workman et al., (Genome Biol. 2002 3, 1-16). In certain embodiments, the numerical value associated with a feature signal is converted into a log number, either before or after normalization occurs.

The process of normalization results in a series of sets of normalized data, each data set containing normalized numerical values corresponding to the amount of the distinguishable signals for a single feature.

In many embodiments of the invention, the normalized numerical values of a single data set (i.e., the set of data obtained from a single feature in all channels) are averaged, and a variability score is calculated for each normalized data set. In most embodiments, the variability score describes the variability of the individual data values within the data set. In general, if the variability score is high, the data points in a data set are not of a very similar value, and if the variability score is low, the data points in a data set are similar. For example, if two data points in a data set are very different numbers, e.g. 1 and 1000, the variability score may be relatively high, and if the two data points in a data set are very similar, e.g. 100 and 102, the variability score may be relatively low. In many embodiments of the invention, variability scores are between 0 and 1, however any two numbers may set the upper and lower limits for variability. In certain embodiments, the variability score is expressed as a percentage of the mean numerical value, or the variability within each data set may be expressed as a fraction of the mean numerical value.

Variability scores may include a standard deviation, which may be calculated by the following formula:

$$S = \sqrt{\frac{1}{n - 1} {\sum }_{i = 1}^{x} {( {X}_{i} - \overline{X} )}^{2}}$$

where S is the standard deviation, n is the number of samples (for example, the number of array features used), and Xi minus Xbaris the difference of a data value from the mean data value for a data set.

Variability scores may include a coefficient of variation, which is the standard deviation of data points in a data set, divided by the mean value of the normalized data. In most embodiments, therefore, each data set is associated with a numerical value that is associated with the variability of data within each data set. This numerical value is an indicator of the confidence that the data within a data set is reliable. A data set that has a coefficient of variation (measured on a scale of 0 to 1.0) that is less than about 0.05, less than about 0.10, less than about 0.15, less than about 0.20, less than about 0.25, less than about 0.30, less than about 0.35, less than about 0.35, or less than about 0.40 or less than about 0.50 is usually a data set that is reliable. In certain embodiments, the data set contains both a mean value and a variability score.

In many embodiments, the reliability of a data point may be determined by model experiments, allowing threshold reliability value to be determined prior to the analysis of any experimental data. In certain embodiments, the reliability value may “tunable”, ie., changeable by a user based on the experimental procedure. In these embodiments, signal variation for a model sample is analyzed to determine an expected signal variation for a probe. Experimental signals may then be compared to the expected noise, i.e., variability, and the reliability of the experimental signal determined, based on whether the experimental signal variation is expected. In other words, model experiments may be performed with a complex sample to determine an expected noise model, to which the observed noise of an experimental sample may be compared. This may be done by: (1) analyzing a given sample in a labeling experiment using more than one dye, and (2) analyzing test experimental models (e.g. gene knockout models). Spiked transcripts at different concentrations may be used to calculate limits of detection, for example a 1.5 fold change. One can then determine the standard deviation (SD), standard error (SE) or coefficient of variation (CV) for the data sets. In some embodiments, one can compare the experimental noise to expected variation to determine if system noise is is unacceptable. For a training model one may examine at intensity of signals (+or −5% or 10% on either side of distribution) of related or unrelated probe sequences to determine reliability score. Such models may use a standard protocol and a platform specific experiment as used in the experiment. If the experimental deviations are greater than prior expected deviations based on model experiments, the experimental datapoint may be “noisy” or unreliable, either due to noise of the system and labeling, etc. or due to differences in nucleic acid concentration. This allows one to estimate the “callability” or reliability of the datapoint in this method of analysis. Following training, the model may be tested on a independent blind test set to evaluate performance of the model and level of statistical error (type I and II) it generates. Thus, tunable data rejection and acceptance criterion can be set by user.

A variability score finds use in many different analysis methods that require a measure of the level of confidence of a data point. In some embodiments, a variability score may be used to provide a “data cut-off”, where data sets that have a coefficient of variation (measured on a scale of 0 to 1.0) that is more than about 0.40, more than about 0.45, more than about 0.50, more than about 0.55, more than about 0.60, more than about 0.65, more than about 0.70, more than about 0.75, more than about 0.80 or more than about 0.90 are deemed “unreliable” and are removed from the data group or flagged as unreliable. As such, data from analysis of signals may be “filtered” to include data sets that have acceptable variability, giving a researcher more confidence in their data.

Alternatively, variability scores may be used in methods for comparing the signals of two features on a single array. For example, data sets for two different features may be compared to determine the relative amounts of signal for two features. In this case, variability scores would facilitate the an assignment of a probability, based on the levels of variation within each data set, that the determined relative amounts are significant. For example, a comparison of two data sets with a 10% difference in mean values of the normalized data sets, but having a high variability score, may indicate that the 10% difference is not significant.

Alternatively, variability scores may be used in methods for comparing data sets from two or more different experiments. For example, in a comparison of signal values of two different features, one on each of two different arrays, may be compared to determine the relative levels of signal for two features. In this case, variability scores would facilitate an assignment of a probability, based on the levels of variation within each data set, that the determined relative levels of signal are significant. For example, a comparison of two data sets with a 10% difference in mean values of the normalized data sets, but having a high variability score, may indicate that the 10% difference is not significant.

Programming according to the present invention, i.e., programming that allows one to perform feature extraction as described above, can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithms for carrying out the above described methodology.

### Utility

The subject methods find use in a variety applications, where such applications are generally analyte detection applications in which the presence or amount of a particular analyte, e.g., a nucleic acid, in a given sample is detected at least qualitatively, if not quantitatively. Protocols for carrying out array assays are well known to those of skill in the art and need not be described in great detail here. Generally, the sample suspected of comprising the analyte of interest is contacted with an array under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system such as a fluorescent label present on the analyte, etc., where detection includes scanning with an optical scanner. The presence or amount of the analyte in the sample is then deduced from the detection of binding complexes on the substrate surface.

Specific analyte detection applications of interest include hybridization assays in which the methods of the subject invention are employed. In these assays, a sample of target nucleic acids is first prepared, where preparation includes labeling of the target nucleic acids with at least two labels, e.g., members of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: pseudo single color genomic hybridization, gene discovery assays, differential gene expression analysis assays; nucleic acid sequencing assays, mutation detection, and the like. The subject methods find particular use for determining the presence of deletions, insertions or rearrangements in a genome, increased nucleic acid copy number in a genome, and other genome alterations. The methods may be used to identify such a genome alteration in an organisms that may be heterozygous or homozygous for the alteration. Such methods usually involve hybridization of an entire genome with an array of probes. For example, in a single experiment, if signals from two independent probes, i.e., probes for different or non-overlapping regions of a target sequence, are reliable (e.g., have high “callability”) but are at significantly different relative levels (in some embodiments, in relation to a known control), an alteration in relative concentration of a genomic fragments corresponding to the probes is indicated. Such an alteration in relative concentration of the genomic fragment indicates that that genomic fragment is altered (e.g., deleted, inserted, present in multiple copies, etc.) in an organisms genome. References describing methods of using arrays in various applications include U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992—the disclosures of which are herein incorporated by reference.

The subject methods find particular use in applications in which a global reference sample, i.e. a suitable control sample, is not available. For example, if only one sample is available, if a suitable “baseline” control (e.g. time “0” in a developmental timecourse experiment) is not available, or if gene expression in a single group of cells or tissue is to be analyzed, a global reference sample may not be available.

In using an array in connection with the methods according to the present invention, the array will typically be exposed to a dual- or triple-labeled sample (such as a fluorescently labeled analyte, e.g., protein or nucleic acid containing sample) and the array then read. Binding complexes on the surface of the array are detected by determining the location and intensity of resulting fluorescence at each feature of the array. Once read, array scans are subject to image analysis and feature extraction to obtain at least two numerical data points for each feature of the array, and this data is analyzed to yield information on the amount of a particular nucleic acid in a sample of nucleic acids, if any.

In any case, results from reading an array may be raw results (such as fluorescence intensity readings for each feature in two or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing). Stated otherwise, in certain variations, the subject methods may be performed in a remote location to scanning. The data may be transmitted to the remote location for further evaluation and/or use. Any convenient telecommunications means may be employed for transmitting the data, e.g., facsimile, modem, internet, etc.

### Kits

Kits for use in connection with the subject invention may also be provided. Such kits preferably include at least a computer readable medium including programming as discussed above and instructions. The instructions may include installation or setup directions. The instructions may include directions for use of the invention with options or combinations of options as described above. In certain embodiments, the instructions include both types of information.

Providing the software and instructions as a kit may serve a number of purposes. The combination may be packaged and purchased as a means of upgrading feature extraction software. Alternately, the combination may be provided in connection with new software. In many embodiments, the instructions will serve as a reference manual (or a part thereof) and the computer readable medium as a backup copy to the preloaded utility.

Kits may alternatively or in addition include reagents such as oligo dT primer, random 6-9 mer primers, Cy-3, Cy-5, dNTPs, NTPs, reverse transcriptase, T7 RNA polymerase, 5-(3-aminoallyl)-2′-deoxyuridine 5′-triphosphate, buffers, etc., for labeling a analyte sample, e.g. a nucleic acid with two or more distinguishable detectable labels, and instructions for using the kit to label an analyte sample and determine the amount, if any, of an analyte in the sample using the methods described above.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or world wide web. Some form of access security or identification protocol may be used to limit access to those entitled to use the subject invention. As with the instructions, the means for obtaining the instructions and/or programming is generally recorded on a suitable recording medium.

In addition to the subject feature extraction software and instructions, the kits may also include one or more reference scans, e.g., two or more reference array scans for use in testing the software after software installation.

### EXPERIMENTAL

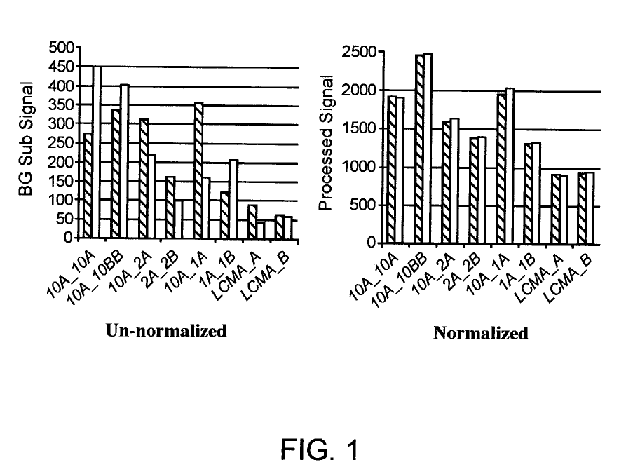

The coefficient of variation (CV) for a feature on a single array was calculated based on standard deviation over the mean of the two intensity measurement (red and green). This was done using the dual scanner (dual laser) detection system. As in FIG. 1, samples with different input RNA have different BG sub signals (left hand graph), but following lowess normalization the processed signal for both channels are balanced (right hand graph). The normalization was performed by feature extraction software. A second normalization (lowess normalization or 1/slope linear correction) can be used across diverse samples used as targets.

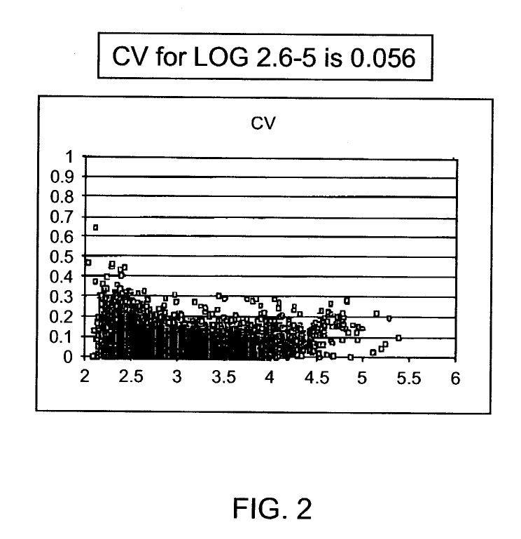

FIG. 2 is a scatter plot showing coefficient of variation for data obtained from an 8,400 element array using 5 microgram human kidney total RNA, with mean BG Sub signals of 1000 and processed signal of 7000. The average coefficient of variation is (6%), and a coefficient of variation is obtained for every data set.

It is evident from the above discussion that the subject invention provides an important breakthrough in the analysis of microarray data. Specifically, the subject invention allows one to assign a variability score, e.g., a coefficient of variation, to data obtained from microarray experiments. This variability score may be used to, e.g., filter out or label data points that are unreliable, giving a researcher the ability to determine the confidence that a certain data point is correct. The subject methods may be used to verify that dye stability or labeling bias does not skew data, providing a significant advantage over methods in which samples are only labeled with a single dye. Also, because the subject methods do not rely on binding competition between two differently labeled nucleic acid populations, the subject methods provide more reliable results. Signal levels may be equal or adjusted by normalization, e.g., lowess normalization. Accordingly, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of determining the amount of an analyte in a sample, said method comprising:
(a) contacting said sample with a probe for said analyte under conditions sufficient for a probe/analyte complex to be produced between said probe and said complex if present in said sample, wherein said sample is labeled with first and second distinguishable detectable labels; and
(b) identifying the amount of said first and said second detectable labels in any resultant probe/analyte complex to determine the amount of said analyte in said sample.

2. The method of claim 1, wherein said analyte is a nucleic acid and said probe/analyte complex is a duplex nucleic acid.

3. The method of claim 1, wherein said analyte and said probe are polypeptides that specifically bind to each other.

4. The method of claim 1, wherein said amount is a qualitative amount.

5. The method of claim 4, wherein said qualitative amount is a relative amount.

6. The method of claim 1, wherein said amount is a quantitative amount.

7. The method of claim 1, wherein said sample comprises a mixture of analytes labeled with said first and second distinguishable detectable labels.

8. The method of claim 1, wherein said labels are fluorescent labels.

9. The method of claim 1, wherein said fluorescent labels are cyanine compounds.

10. The method of claim 9, wherein said first detectable label is cyanine-3 and said second detectable label is cyanine-5.

11. The method of claim 2, wherein said probe is a nucleic acid that is greater than about 20 nucleotides in length.

12. The method of claim 1, wherein said identifying step (b) comprises measuring the amount of fluorescence of said first and said second detectable labels.

13. The method of claim 1, wherein said identifying step (b) comprises averaging the amounts of said first and said second detectable labels.

14. The method of claim 1, wherein said identifying step (b) further comprises normalizing said amounts of said first and said second detectable labels.

15. The method of claim 1, wherein said contacting step (a) comprises incubating a labeled analyte sample with an array of probes for different gene products.

16. The method of claim 1, further comprising assessing the variability of the amount of said first and said second detectable labels in any resultant probe/analyte complex.

17. The method of claim 16, further comprising calculating a variability score so assess said variability.

18. The method of claim 17, wherein said variability score provides a score of the reliability of the determined amount of said analyte in said sample.

19. A method of determining the relative amount of an analyte in two samples, said method comprising:
(a) determining the amount of said analyte in a first sample by the method of claim 1;
(b) determining the amount of said analyte in a second sample by the method of claim 1; and
(c) comparing the amounts of said analyte in said samples to determine the relative amount of said analyte in said two samples.

20. The method of claim 19, wherein said first and second labeled samples are each contacted with substantially identical probes present on different substrates.

21. The method of claim 20, wherein said substrates are arrays comprising probes for at least 10 gene products.

22. The method of claim 19, wherein said relative amount is normalized relative to controls.

23. The method of claim 19, wherein said determining step (a) comprises obtaining an average analyte amount for said first sample, said determining step (b) comprises obtaining an average analyte amount for said second sample and said comparing step (c) comprises comparing the average analyte amount for the first sample to the average analyte amount level for the second sample.

24. A method of detecting the presence of an analyte in a sample, said method comprising:
(a) contacting a sample suspected of containing said analyte with an array of probes for said analyte, wherein said sample is labeled with a first and a second distinguishably detectable labels;
(b) detecting any binding complexes on the surface of the said array by determining the amounts of said first and second distinguishably detectable labels to obtain binding complex data; and
(c) determining the presence of said analyte in said sample using said binding complex data.

25. The method according to claim 24, wherein said analyte is a nucleic acid and said array is an array of nucleic acid probes.

26. A method comprising transmitting data obtained from a method of claim 24 from a first location to a second location.

27. A method according to claim 26, wherein said second location is a remote location.

28. A method comprising receiving data representing a result of a reading obtained by the method of claim 24.

29. A hybridization assay comprising the steps of:
(a) contacting at least one target nucleic acid sample labeled with first and second distinguishable detectable labels with a nucleic acid array to produce a hybridization pattern for said nucleic acid sample; and
(b) analyzing said hybridization pattern for each detectable label to produce data on the amounts of said target nucleic acid in said sample.

30. The method according to claim 29, wherein said method further comprises washing said array prior to said detecting step.

31. A computer-readable medium comprising a program that determines the level of an analyte in a sample by averaging the levels of two distinguishable labels in complex comprising a probe for said analyte.

32. The computer readable medium of claim 31, further comprising:
a program for determining the relative levels an analyte in two or more analyte samples.

33. The computer readable medium of claim 31, wherein said levels of two distinguishable labels are determined by quantifying the average brightness of a pixilated image representing the level of fluorescence of a probe/analyte complex for each label.

34. A kit comprising said computer readable medium of claim 31, further comprising:
instructions for performing the method of claim 1.

35. A kit comprising:
(a) reagents for labeling analyte sample with a first and a second detectable label; and
(b) instructions for using the kit in the method of claim 1.

36. The kit according to claim 35, wherein said analyte sample is a nucleic acid sample.
